(19) United States
(12) Patent Application Publication
(10) Pub. No.: US 2026/0014081 A1
(43) Pub. Date: Jan. 15, 2026

(54) Pharmaceutical composition and method for enhancing solubility of poorly soluble active pharmaceutical ingredients

(71) Applicant: MERCK PATENT GMBH, Darmstadt (DE)

(72) Inventors: Thomas KIPPING, Darmstadt (DE); Jonas LINDH, Uppsala (SE); Julian QUODBACH, Uppsala (SE)

(73) Assignee: MERCK PATENT GMBH, Darmstadt (DE)

(21) Appl. No.: 18/881,183

(22) PCT Filed: Jul. 3, 2023
(86) PCT No.: PCT/EP2023/068142

§ 371 Date: Jan. 3, 2025

(30) Foreign Application Priority Data
Jul. 6, 2022 (EP) 22183235.5
Nov. 17, 2022 (EP) 22208141.6

(51) Int. Cl. A61K 9/16

(52) U.S. Cl. CPC A61K 9/1635; A61K 9/1694

(57) ABSTRACT

The present disclosure generally relates to the use of polyvinyl alcohol (PVA) in additive manufacturing technologies and techniques. More specifically, the present disclosure relates to the use of PVA in a selective laser sintering (SLS) method to additively manufacture an object, in particular a pharmaceutical dosage form.

### TECHNICAL FIELD

The present disclosure generally relates to the use of polyvinyl alcohol (PVA) in additive manufacturing technologies and techniques. More specifically, the present disclosure relates to the use of PVA in a selective laser sintering (SLS) method to additively manufacture an object, in particular a pharmaceutical dosage form.

### BACKGROUND

The use of hydrophilic polymers such as polyvinyl alcohol as an excipient for pharmaceutical compositions has been widely described. WO 2018/083285 A1 discloses powdered PVA having improved properties as a polymer matrix in pharmaceutical compositions comprising active ingredients, especially in compressed tablets forming amorphous solid dispersions with poorly soluble active pharmaceutical ingredients (APIs).

Polyvinyl alcohol is used in amorphous solid dispersions which is a well-known strategy to improve the bioavailability of poorly water-soluble drug substances. Although the amorphous form exhibits higher solubility, it is rather unstable and tends to re-crystallize and precipitate immediately after dissolution or during the pH change while changing from the acidic gastric environment to the more neutral intestine. The re-crystallized fraction of the API cannot be absorbed. Since drug absorption occurs primarily in the intestines, pharmaceutical formulations that do not sustain high concentration of the APIs in an intestinal solution typically yield only minor improvements in bioavailability. The undesirable recrystallization rather reduces the bioavailability of the API. Poor bioavailability is a significant problem encountered in the development of pharmaceutical compositions, particularly those containing an API that is not highly water-soluble.

In J Pharm Sci. 2008; 97(12): 5198-211, polyvinyl alcohol was successfully evaluated to inhibit the crystal formation of model compounds like caffeine. The grade of PVA used for these data is described as polyvinyl alcohol (PVA) with an average molecular weight of 47,000. Another study using PVA to increase the supersaturated state of a model compound tacrolimus is described by Overhoff et al., Effect of Stabilizer on the Maximum Degree and Extent of Supersaturation and Oral Absorption of Tacrolimus Made By Ultra-Rapid Freezing, Pharmaceutical Research. 2008; 25(1): 167-75. Solid dispersions are prepared by ultra rapid freeze drying. The used PVA grade is described as Poly(vinyl) alcohol (PVA, Mw 13,000-23,000, 87-89% hydrolyzed). PVA could be successfully used as a stabilizer. The use of polyvinyl alcohol for hot melt extrusion has previously been described by de Jaeghere et al., Hot-melt extrusion of polyvinyl alcohol for oral immediate release applications, Int J Pharm. 2015; 492(1-2): 1-9. Partly hydrolyzed PVA grades were used to evaluate the use as a carrier for oral immediate release dosage forms. An impact on release rates was observed, but no direct link between hydrolysis degree and supersaturation potential was identified. In Brough et al., Use of Polyvinyl Alcohol as a Solubility Enhancing Polymer for Poorly Water-Soluble Drug Delivery (Part 1), AAPS PharmSciTech Vol. 17, No. 1, p. 176 (Jan. 2, 2016) certain PVA grades including PVA 4-75, PVA 4-88, PVA 4-98, PVA 4-38 by a non-sink gastric transfer dissolution method were investigated.

Selective Laser Sintering (SLS) is one of the most popular additive manufacturing processes that creates a three-dimensional (3D) object layer-by-layer. The process applies layers of powder material on top of each other sequentially, where each layer of powder is sintered or coalesced together with a laser according to the computer aided drawing (CAD) geometry of the part.

SLS is a powder bed based additive manufacturing technique to produce complex three-dimensional parts. In SLS, a rasterized laser is used to scan over a bed of polymer powder, sintering it to form solid shapes in a layer-wise fashion. When the laser beam scans the powder, the powder melts due to the rising temperature, and layer by layer, the final part approaches full density and should result in properties of the bulk material (the polymer). By controlling the energy input it is possible to control the density of the sintered material and to achieve parts ranging from highly porous to almost full dense. In theory, every thermoplastic polymer that can be transformed into a powder form can be processed via this technique, but the reality is that every material behaves differently, often unpredictably, during melting, coalescence, and consolidation, and often requires unique SLS processing parameters. The bed temperature and laser energy input, for example, can be selected based on the processing window of the polymer's thermal profile as well as its energy absorption. Laser parameters can also be selected based on the powder's particle size and shape.

There are different types of polymer particles that are generally used in the SLS process. Semi-crystalline resins such as polyamides including PA12, PA11, and PA6, polylactic acid (PLA), polyether ether ketone (PEEK), polyethylene (PE), polypropylene (PP), and others are used. The most common polymer powder employed is polyamide PA12. The common name for polyamide is nylon. For example polyamide PA12 is also known as nylon 12, polyamide PA6 is also known as nylon 6. A layer-upon-layer structure is formed by sintering the polymer particles together with a laser above the melting point of the polymer according to the CAD geometry file of the part.

For the use of polyvinyl alcohol as pharmaceutical excipient in selective laser sintering there is currently comparably little experience. In Basit et al. International Journal of Pharmaceutics 529 (2017) 285-293 Kollicoat IR, which is a copolymer of 75% polyvinyl alcohol and 25% polyethylene, was used in a SLS process with paracetamol as active pharmaceutical ingredient. Yang et al. International Journal of Pharmaceutics 593 (2021) 120127 disclosed PVA as one of several excipients for SLS printing. So far, the suitability of different PVA grades (viscosity and hydrolysis degree) is unknown.

There is still a need for polyvinyl alcohols as pharmaceutical excipients having improved properties for the method of selective laser sintering.

### SUMMARY OF THE INVENTION

It was surprisingly found that polyvinyl alcohol can be used in a process for selective laser sintering of sinter powder to form a pharmaceutical dosage form. It was further found that polyvinyl alcohol having a hydrolysis degree of 70% to 90%, and a viscosity of a 4% solution at 20° C. of 3 mPas to 8 mPas is particularly suitable for selective laser sintering.

Unexpectedly, it was shown that there is an optimum range of hydrolysis degree and viscosity which leads to pharmaceutical dosage form with beneficial properties, such as a faster API release and/or an improved amorphization of the API compared to commonly used PVA grades. The PVA grades within this optimum range assure and stabilize the release and supersaturation of APIs with different 3D printing parameters. Furthermore, it has been shown that the amorphous form of the API can be long-term stabilized in the pharmaceutical dosage forms, e.g. more than 6 month. In a preferred embodiment the long-term stabilization is measured at 40° C./dry conditions, 25° C./60% RH, 30° C./65% RH, 30° C./75% RH or 40° C./75% RH for 3 or 6 month, wherein more than 80%, 85%, 90%, 92%, 95%, 97%, 98% or 99% of the API is still present in its amorphous form.

In a preferred embodiment of the invention, the polyvinyl alcohol has a hydrolysis degree of 80% to 90%, and a viscosity of a 4% solution at 20° C. of 3 mPas to 4 mPas.

In another aspect, the invention provides a process for producing a pharmaceutical dosage form by selective laser sintering of sinter powder, comprising the step of
(a) providing a sinter powder comprising at least one active pharmaceutical ingredient and polyvinyl alcohol having a hydrolysis degree of 70% to 90%, and a viscosity of a 4% solution at 20° C. of 3 mPas to 8 mPas and
(b) operating a selective laser sintering apparatus that selectively fuses layers of the sinter powder to produce the pharmaceutical dosage form.

A further aspect of the invention concerns a sinter powder for selective laser sintering, comprising at least one active pharmaceutical ingredient and polyvinyl alcohol having a hydrolysis degree of 70% to 90%, and a viscosity of a 4% solution at 20° C. of 3 mPas to 8 mPas. The sinter powder can comprise further excipients and/or light-absorbing materials.

A further aspect of the invention concerns a pharmaceutical dosage form obtainable by a process as mentioned above, in particular a pharmaceutical dosage form produced by selective laser sintering of sinter powder, wherein the sinter powder comprises at least one active pharmaceutical ingredient and polyvinyl alcohol having a hydrolysis degree of 70% to 90%, and a viscosity of a 4% solution at 20° C. of 3 mPas to 8 mPas.

### DETAILED DESCRIPTION OF THE INVENTION

An embodiment of the invention is the use of polyvinyl alcohol in a process for selective laser sintering of sinter powder to form a pharmaceutical dosage form, wherein the sinter powder comprises at least one active pharmaceutical ingredient and polyvinyl alcohol having a hydrolysis degree of 70% to 90%, and a viscosity of a 4% solution at 20° C. of 3 mPas to 8 mPas.

According to the invention selective laser sintering is a process in which a laser beam is used to sinter and/or melt a powder bed filled with a powder mixture containing polymer by scanning the laser according to the cross-section of a digital model. A polymer version of the digital model is produced in a layer-by-layer fashion by laser scanning successive layers of powder mixture. The process will require a selective laser sintering printer equipped with a laser source and a galvanometric system for scanning the laser on the powder bed surface or, alternatively, a xy motion system where the actual laser source is moved to scan the powder bed. The printer must also provide a powder application system to spread the powder in layers as well as some heating capabilities to heat the build chamber and the surface of the powder bed.

According to the invention, the powder mixture used is prepared by mixing PVA, API and optionally further pharmaceutically acceptable excipients. The PVA is first sieved through a 300 micron sieve and the material which pass through the sieve are mixed with excipients and API and mixed in a turbula mixer for 30 min. The resulting mixture is then sieved through a 300 micron sieve and the material which pass through the sieve is loaded into the printer.

After loading the mixture, the printer is preheated to a set temperature below the Tg of the PVA and the printing process is initiated. In the printing process parameters such as chamber and print bed temperature are set to appropriate values obtained via experimental studies to provide printed tablets with desirable properties with respect to mechanical and morphological properties. Other parameters influencing the process are laser energy input and layer height of each applied layer. The laser energy input can be controlled in a number of ways depending on which type of printer is used and usually via adjusting laser scanning speed, hatching space (distance between scanned laser lines) or by adjusting the energy output by the laser.

Once the printing process is finalized the printed tablets are allowed to slowly cool down in the printer before being removed and cleaned from surrounding, unsintered powder.

Polyvinyl alcohol (PVA) is a synthetic water-soluble polymer that has the idealized formula [CH₂CH(OH)]ₙ. It possesses good film-forming, adhesive, and emulsifying properties. PVA is prepared from polyvinyl acetate, where the functional acetate groups are either partially or completely hydrolysed to alcohol functional groups. If not completely hydrolysed, PVA is a random copolymer consisting of vinyl alcohol repeat units —[CH₂CH(OH)]— and vinyl acetate repeat units —[CH₂CH(OOCCH₃)]—. The polarity of PVA is closely linked to its molecular structure. The hydrolysis degree and the molecular weight determine the molecular properties of PVA. As the degree of hydrolysis of acetate groups increases, the solubility of the polymer in aqueous media and also crystallinity and melting temperature of the polymer increase. However, at high hydrolysis degrees over 88%, the solubility of PVA decreases again. PVA is generally soluble in water, but almost insoluble in almost all organic solvents, excluding, in some cases, ethanol.

The typical PVA nomenclature indicates the viscosity of a 4% solution at 20° C. and the degree of hydrolysis of the polymer. For example, PVA 3-83 is a PVA grade with a viscosity of 3 mPas that is 83% hydrolysed, i.e. having 83% of vinyl alcohol repeat units and 17% of vinyl acetate repeat units. A skilled person is aware that a hydrolysis grade of 83% and a viscosity of 3 mPas encompasses calculated hydrolysis grades of 82.50% to 83.49% and calculated viscosities of 2.50 mPas to 3.49 mPas % according to common rounding methods. Viscosity according to the invention is measured as stated in USP 39 under Monograph “Polyvinyl Alcohol” with the method Viscosity-Rotational Method (912).

The degree of hydrolysis according to the invention is measured by determining the saponification value of the Polyvinyl Alcohol, e.g. as stated in USP 39 under Monograph “Polyvinyl Alcohol” under “Degree of Hydrolysis”:

### Sample: 1 g of Polyvinyl Alcohol, Previously Dried at 110° to Constant Weight

### Analysis

Transfer the Sample to a wide-mouth, 250-ml conical flask fitted by means of a suitable glass joint to a reflux condenser. Add 35 ml of dilute methanol (3 in 5), and mix gently to ensure complete wetting of the solid. Add 3 drops of phenolphthalein TS, and add 0.2 N hydrochloric acid or 0.2 N sodium hydroxide if necessary, to neutralize. Add 25.0 ml of 0.2 N sodium hydroxide VS, and reflux gently on a hot plate for 1 h. Wash the condenser with 10 ml of water, collecting the washings in the flask, cool, and titrate with 0.2 N hydrochloric acid VS. Concomitantly perform a blank determination in the same manner, using the same quantity of 0.2 N sodium hydroxide VS.

### Calculation of Saponification Value:

### Calculate the Saponification Value:

$$Result = [ ( {V}_{B} - {V}_{S} ) \times N \times {M}_{r} ] / W$$

V_{B}=volume of 0.2 N hydrochloric acid VS consumed in the titration of the blank (ml)
V_{S}=volume of 0.2 N hydrochloric acid VS consumed in the titration of the Sample solution (ml)
N=actual normality of hydrochloric acid VS
Mᵣ=molecular weight of potassium hydroxide, 56.11
W=weight of the portion of Polyvinyl Alcohol taken (g)

### Calculation of Degree of Hydrolysis:

### Calculate the Degree of Hydrolysis, Expressed as a Percentage of Hydrolysis of Polyvinyl Acetate:

$$Result = 100 - [ 7.84 \times S / ( 100 - 0.075 \times S ) )$$

S=saponification value of the Polyvinyl Alcohol

The use of PVA grades according to the invention is of interest for the formulation of solid oral pharmaceutical dosage forms with an instant, immediate or prolonged API release.

Preferred PVAs have a hydrolysis degree of 70% to 90%, and a viscosity of a 4% solution at 20° C. of 3 mPas to 5 mPas, more preferably a viscosity of a 4% solution at 20° C. of 3 mPas to 4 mPas, most preferably a viscosity of a 4% solution at 20° C. of 4 mPas.

In a further embodiment of the invention, the polyvinyl alcohol has a hydrolysis degree of 70% to 90%, preferably 80% to 90% and a viscosity as mentioned above.

In a further embodiment of the invention, the polyvinyl alcohol has a hydrolysis degree of 80% to 90% and a viscosity of a 4% solution at 20° C. of 3 mPas or a hydrolysis degree of 80% to 90% and a viscosity of a 4% solution at 20° C. of 4 mPas.

In a further embodiment of the invention, the polyvinyl alcohol is PVA 3-80, PVA 3-82, PVA 4-88 or PVA 5-74, preferably PVA 3-80, PVA 3-82 or PVA 4-88, more preferably PVA 4-88.

In a further embodiment of the invention, the polyvinyl alcohol is PVA 3-82, PVA 4-88 or PVA 5-74, more preferably PVA 3-82 or PVA 4-88, most preferably PVA 3-82.

The above mentioned embodiments for APIs and/or PVA grades apply equally for the use of polyvinyl alcohol in a process for selective laser sintering of sinter powder, the process for producing a pharmaceutical dosage form by selective laser sintering of sinter powder, the sinter powder for selective laser sintering and the pharmaceutical dosage form produced by selective laser sintering of sinter powder as mentioned above.

A further embodiment of the invention is a sinter powder for selective laser sintering, comprising at least one active pharmaceutical ingredient and polyvinyl alcohol having a hydrolysis degree of 70% to 90%, and a viscosity of a 4% solution at 20° C. of 3 mPas to 8 mPas.

For the avoidance of doubt, the processes according to the invention include selective laser sintering with any of the PVAs, PVA specifications or PVA grades as defined below.

In a further embodiment of the invention, the sinter powder and the pharmaceutical dosage form may comprise further pharmaceutically acceptable excipients. Pharmaceutically acceptable excipients comprise flow control agents, such as silicon dioxide, fillers, plasticizers, surfactants, light-absorbing material, such as ruby red or Candurin pigments and other suitable components that are well known to those skilled in the art.

Depending on the wavelength of the light emitted by the laser, a light-absorbing material (pigment) which absorbs light at the wavelength emitted may be required. These light-absorbing materials can contain transition metals for absorption at around 450 nm or carbon for a wider range covering the visible—and near IR range. Light absorption is a process by which light is absorbed and converted into energy. When light is absorbed heat is generated. So the selective absorption of light by a particular material occurs because the frequency of the light wave matches the frequency at which electrons in the atoms of that material vibrate.

Light-absorbing materials are all materials suitable for the SLS method as described above and known to the skilled person in the art. Preferably light-absorbing materials which have been demonstrated to work at 455 nm laser irradiation are used, e.g. Candurin NXT, Ruby Red, Candurin Gold Sheen, Aluminum Lake, activated carbon (also works at 808 nm) or iron oxide (Fe2O3). More preferably ruby red is used.

Carbon dioxide laser emitting at around 10 microns will usually not require addition of light-absorbing materials as C—H bonds absorb energy will at this wavelength and this type of bonds can be found in most polymers.

For the avoidance of doubt further pharmaceutically acceptable excipients as defined above are not needed for the beneficial properties according to the invention. Yet those excipients can be used for other purposes, e.g. to optimize the process of manufacturing of the pharmaceutical composition or oral dosage form according to the invention.

Furthermore, the pharmaceutical composition according to the invention may comprise additional pharmaceutically acceptable hydrophilic or lipophilic polymers.

In a preferred embodiment the sinter powder further comprises a light-absorbing material.

As used herein, the phrase “pharmaceutically acceptable” refers to all excipients, polymers, compounds, solvents, dispersion media, flow control agents, carriers, coatings, active agents, isotonic and absorption delaying agents, and the like that do not produce an allergic or similar untoward reaction when administered to humans in general. The use of such material in pharmaceutical compositions is well known in the art.

In a preferred embodiment the particle size of the sinter powder has a D50 of 200 μm or lower. Preferably, the particle size (D50) of the sinter powder is between 20 μm and 200 μm, 20 μm and 150 μm or 20 μm and 100 μm.

In a preferred embodiment the particle size of the PVA has a D50 of 200 μm or lower. Preferably, the particle size (D50) of the PVA is between 20 μm and 200 μm, 20 μm and 150 μm or 20 μm and 100 μm.

In a further preferred embodiment the particle size of the PVA has a D90 of 250 μm or lower. Preferably, the particle size (D90) of the PVA is between 100 μm and 250 μm, more preferably between 140 μm and 220 μm.

A further embodiment of the invention is a process for producing a pharmaceutical dosage form by selective laser sintering of sinter powder, comprising the step of
(a) providing a sinter powder comprising at least one active pharmaceutical ingredient and polyvinyl alcohol having a hydrolysis degree of 70% to 90%, and a viscosity of a 4% solution at 20° C. of 3 mPas to 8 mPas and
(b) operating a selective laser sintering apparatus that selectively fuses layers of the sinter powder to produce the pharmaceutical dosage form.

For the avoidance of doubt, the processes according to the invention include selective laser sintering with any of the PVAs, PVA specifications or PVA grades as defined below.

A further embodiment of the invention is a pharmaceutical dosage form obtainable by the process for producing a pharmaceutical dosage form by selective laser sintering of sinter powder as described above.

In a further embodiment the pharmaceutical dosage form is produced by selective laser sintering of sinter powder wherein the sinter powder comprises at least one active pharmaceutical ingredient and polyvinyl alcohol having a hydrolysis degree of 70% to 90%, and a viscosity of a 4% solution at 20° C. of 3 mPas to 8 mPas.

For the avoidance of doubt, the pharmaceutical dosage forms according to the invention include pharmaceutical dosage forms with any of the PVAs, PVA specifications or PVA grades as defined below.

The API is a biologically active agent. The API may be a small molecule in form of a weak base, a weak acid or a neutral molecule and may be in the form of one or more pharmaceutically acceptable salts, esters, derivatives, analogues, prodrugs, and solvates thereof. The sinter powder and the pharmaceutical dosage form may comprise more than one API. In one embodiment the API is poorly soluble or a lipophilic API.

As used herein, the terms “poorly soluble API”, “poorly water-soluble API” and “lipophilic API” refer to an API having a solubility such that the highest therapeutic dose of the particular API to be administered to an individual cannot be dissolved in 250 ml of aqueous media ranging in pH from 1 to 8 following the definition of low solubility according to the Biopharmaceutics Classification System (BCS) classes 2 and 4. Poorly soluble APIs with weakly basic or weakly acidic characteristics have a pH-dependent solubility profile and can have a wide range of solubility in the aqueous environment of the gastrointestinal tract. APIs falling under BCS classes 2 or 4, respectively, are well known to persons skilled in the art.

In one embodiment the API is a weakly basic API. As used herein, the term “weakly basic API” refers to a basic active pharmaceutical ingredient (API) wherein the basic API does not completely ionize in water.

The at least one active pharmaceutical ingredient (API) according to the invention may be dispersed in the polyvinyl alcohol forming an amorphous solid dispersion.

A further embodiment of the invention is the use of polyvinyl alcohol in a process for selective laser sintering of sinter powder to form a pharmaceutical dosage form, wherein an amorphous solid dispersion of at least one active pharmaceutical ingredient in the polyvinyl alcohol is formed and wherein the polyvinyl alcohol has a hydrolysis degree of 70% to 90%, and a viscosity of a 4% solution at 20° C. of 3 mPas to 8 mPas.

As used herein, the term “amorphous solid dispersion” is a dispersion of at least one amorphous API in a polymer matrix. Preferably, the amorphous API is distributed in a molecularly dispersed state within the polymer matrix. In this case, the solid dispersion is a solid solution. Upon dissolution, formulations comprising an amorphous solid dispersion can reach higher solubilities in aqueous media than the crystalline API.

The API included in the pharmaceutical dosage form of the present invention has a sufficient amount to be therapeutically effective. For a given API, therapeutically effective amounts are generally known or readily accessible by persons skilled in the art. Typically, the API may be present in the pharmaceutical dosage form in a weight ratio of API to polyvinyl alcohol of 0.1:99.1 to 60:40, preferably 1:99 to 50:50, more preferably 5:95 to 40:60 and most preferably 10:90 to 30:70.

It was found that PVAs having a degree of hydrolysis and a viscosity in the ranges as mentioned above show a surprisingly good performance when used in a process for selective laser sintering of sinter powder to form a pharmaceutical dosage form comprising an active pharmaceutical ingredient compared to PVAs outside the ranges as mentioned above.

It was unexpectedly found that selective laser sintering of sinter powder comprising an active pharmaceutical ingredient and PVAs having the above-identified viscosity and hydrolysis grades lead to pharmaceutical dosage forms that
show a faster and higher API release and
have an improved amorphization of the API.

The improved amorphization of pharmaceutical dosage forms manufactured by SLS printing with a PVA having a degree of hydrolysis and a viscosity in the ranges as mentioned above can be seen when Powder X-ray diffraction (PXRD) data and/or differential scanning calorimetry (DSC) data are compared to pharmaceutical dosage forms manufactured by SLS printing with a PVA outside the ranges as mentioned above under the same process conditions. A preferred PVA is polyvinyl alcohol having a hydrolysis degree of 70% to 90%, and a viscosity of a 4% solution at 20° C. of 3 mPas to 8 mPas, more preferrable the polyvinyl alcohol having a hydrolysis degree of 80% to 90%, and a viscosity of a 4% solution at 20° C. of 3 mPas to 4 mPas. Preferred PVA grades are PVA 3-80, PVA 3-82, PVA 4-88 or PVA 5-74. The best amorphization results have been demonstrated with PVA 4-88 and PVA 3-82, preferably PVA 4-88.

Furthermore, PVAs having the above-identified viscosity and hydrolysis grades assure and stabilize the release and supersaturation of APIs, in particular poorly soluble APIs, in aqueous media thereby preventing crystallization and phase separation. Since a low water solubility of an API in general accompanies a low bioavailability after its administration in a pharmaceutical preparation, the compositions according to the invention also contribute to improving the bioavailability of poorly water-soluble APIs, and particularly weakly basic APIs.

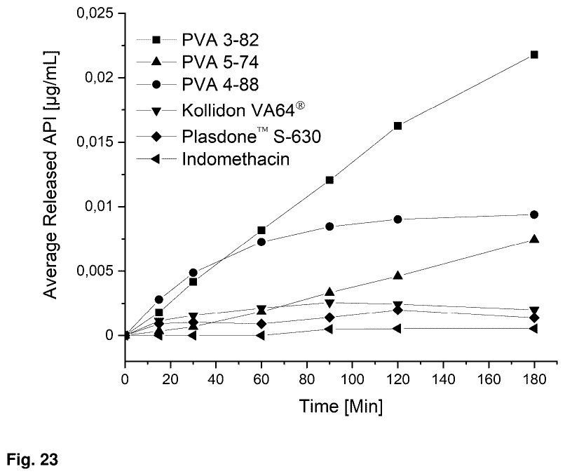

It has been shown that pharmaceutical dosage forms manufactured by SLS printing with a PVA having a degree of hydrolysis and a viscosity in the ranges as mentioned above have a significantly faster and more pronounced API release. Dissolution experiments prove that PVA grades of the present invention, e.g. PVA 3-82, PVA 5-74 and PVA 4-88, are particularly suitable as polymers for that purpose. PVA 3-82 has the highest API release compared to the other PVA grades (FIG. 23) and provides a solubility enhancement compared to the crystalline form of indomethacin. This is particularly surprising, since that result could not have been expected from the PXRD and/or DSC data.

Furthermore, PVAs having the above-identified viscosity and hydrolysis grades have an improved surface quality with a smooth surface finish, which can be seen with scanning electron microscope (SEM) method. They differ in surface area which can be measured by BET (gas adsorption), tablet hardness and porosity which can be measured by μCT measurements.

PVAs having the above-identified viscosity and hydrolysis grades furthermore show improved API stability.

As used herein, “bioavailability” is a term meaning the degree to which an API becomes available to the target tissue after being administered to the body of a patient.

After dissolving pharmaceutical dosage forms of the present invention an improved supersaturation is overserved and the API is kept better in solution. Furthermore, the API can be better incorporated into the PVA matrix during the sintering process.

Having entered the gastrointestinal tract, the pharmaceutical dosage form swells and disintegrates in the aqueous environment of the gastrointestinal fluids thereby releasing the API. While a salt form of a weakly basic API may show improved initial aqueous concentration in the acidic gastric fluid, the weakly basic API rapidly converts to the free base form in the more neutral intestinal fluid where the free base-form of the API has a significantly lower equilibrium concentration. PVAs according to the invention maintain enhanced concentrations of the API in model solutions simulating acidic and neutral gastrointestinal solutions as compared to commonly used PVAs. Therefore, the pharmaceutical dosage forms according to the invention have the potential to provide enhanced bioavailability of poor solubility APIs. The solubility-improved form of the API in the presence of a PVA grade according to the invention provides a concentration of the API in gastric fluid or simulated gastric fluid that is greater than the concentration of the API provided in the presence of commonly used PVA grades.

### EXAMPLES

### Example 1: SLS Printing with PVA 4-88

### Preparation of Powder Formulation

Indomethacin-loaded powder formulations were prepared according to Table 1. All powder mixtures were sieved using a 315 μm stainless-steel test sieve (VWR International AB, Sweden) and mixed using a Turbula shaker (Turbula T2F shaker, Glen Mills, Inc., Clifton, NJ, US) for 15 min. Candurin Ruby Red and colloidal silica (Aerosil) were added to the formulations in order to enhance the laser energy absorption of the powders and to improve powder flowability during the layer application process, respectively. As polyvinyl alcohol, PVA 4-88 Parteck® MXP (Merck KGaA) was used. The formulations were prepared in large enough batches (approx. 1500 mL) to partially fill the build volume (100×100×100 mm) of a Sintratec Kit SLS 3D printer (Sintratec, Brugg, Switzerland).

TABLE 1
Composition of the prepared powder formulations

| Formulation | PVA 4-88 | Ruby Red | Aerosil | Indomethacin |
|---|---|---|---|---|
| wt. % | 88.5 | 0.5 | 1 | 10 |

### Selective Laser Sintering 3D Printing of Dosage Forms

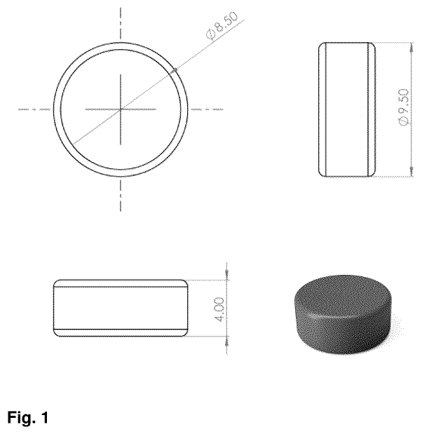

Tablet templates were created and designed in Solidworks 2019 SP05 (FIG. 1), and the obtained standard triangle language file (STL-file) was subsequently prepared for printing in Sintratec software using the process parameters presented in Table 2. The energy density was calculated according the following equation:

$$e = α \frac{4 P}{π \cdot HS \cdot V} = α \frac{4 \cdot 2.3}{π \cdot 0.05 \cdot 200} = α \cdot 0.29 J / {mm}^{2}$$

P—laser power (2.3 W); HS—hatching space (0.05 mm); V—scanning speed (mm/s); α—absorptivity of the powder (coefficient should be measured/calculated for each powder type with respect to absorption at 455 nm).

TABLE 2
Process parameters used for each formulation

| | Print bed | Chamber | | Energy | Layer |
|---|---|---|---|---|---|
| | temperature | temperature | Laser scanning | density α | height |
| Batch | (° C.) | (° C.) | speed (mm/s) | (J/mm²) | (μm) |
| 1 | 75 | 70 | 200 | 0.292 | 125 |
| 2 | 75 | 70 | 300 | 0.195 | 125 |
| 3 | 75 | 70 | 400 | 0.146 | 125 |
| 4 | 100 | 70 | 200 | 0.292 | 125 |
| 5 | 100 | 70 | 300 | 0.195 | 125 |
| 6 | 100 | 70 | 400 | 0.146 | 125 |
| 7 | 125 | 70 | 200 | 0.292 | 125 |
| 8 | 125 | 70 | 300 | 0.195 | 125 |
| 9 | 125 | 70 | 400 | 0.146 | 125 |

FIG. 1 is showing a schematic drawing and 3D model of a tablet (4×9.5 mm).

The 3D printing process was further carried out as follows: The prepared powder formulations (Table 1) were placed and packed into the powder reservoir platform (100×100×100 mm) of the SLS 3D printer. A thin layer of the formulation was thereafter spread onto the build platform after which the powder beds were slowly heated to the temperatures specified in Table 2. The sintering process was carried out using a 2.3 W diode (λ=455 nm) in accordance with the models given in the STL-file in a layer-by-layer fashion. A total of 36 tablets were printed per batch, flat to the build platform, using a layer height of 125 μm. Specific values for the laser scanning speed were chosen when printing the different batches and each speed was used at three different print bed temperatures. The finished batches were collected from the build platform at the end of the printing process by sieving. The tablets were additionally de-dusted using pressurized air in order to remove excess powder and stored in sealed containers for further analysis.

### Characterization of Powder Formulations and 3D Printed Dosage Forms

Powder X-ray diffraction (PXRD) diffractograms of the pristine and heat-treated powder formulations as well as the printed dosage forms were collected on a Bruker D8 Advance TwinTwin diffractometer (Bremen, Germany) using Cu-Kα_{1.2} (λ=1.5418 Å) radiation. The instrument was operated at 40 mA and 40 kV, using a step-size of 0.02° and a data collection time of 1 h. Differential scanning calorimetry (DSC) thermograms were obtained on a Mettler Toledo DSC 3+ (Schwerzenbach, Switzerland) using a heating and cooling rate of 10° C. min⁻¹ and nitrogen as purge gas. Repeated heating-cooling measurements were carried out from −40 to 200° C. and from 200 to 10° C. in the first cycle, and from 10 to 200° C. in the following cycles. The dimensions (n=10) and weights (n=30) of the printed tablet were examined using a digital caliper and an analytical balance (Mettler Toledo XS 64 Analytical Balance, Schwerzenbach, Switzerland). Friability tests were carried out in accordance with the European Pharmacopoeia (Ed. 10.0) on approx. 6.5 g of tablets using a Pharmatest PTF E friabilator (Hainberg, Germany) at 25 rpm and for 100 rotations. The tablets were carefully weighed pre- and post-measurement and total weight loss of the tablets (i.e. friability) calculated.

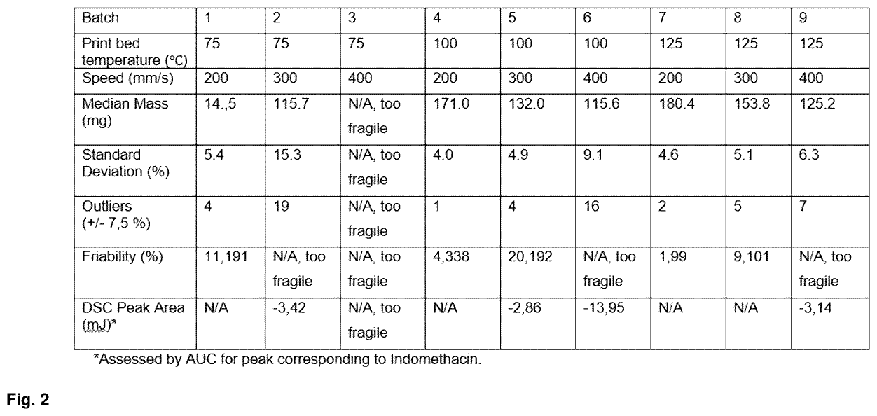

FIG. 2 shows process parameters and characteristics of 3D printed dosage forms.

Traces of crystalline API remaining for batch printed at 75° C. and 300 mm/s whereas 200 mm/s results in fully amorphized API.

In the figures PVA 4-88 is referred to as Parteck MXP.

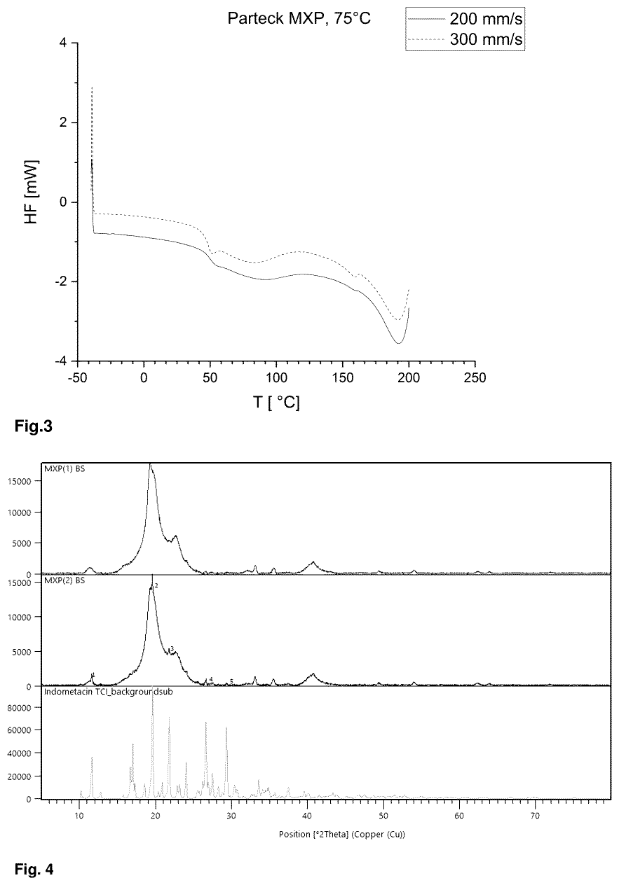

FIG. 3 shows the DSC thermogram for batches printed at 75° C. with PVA 4-88.

FIG. 4 shows diffractograms for batches printed at 75° C. and Indomethacin with PVA 4-88 (the number in parentheses reflects the batch number).

Traces of crystalline API remaining for batch printed at 100° C. and 300 and 400 mm/s whereas 200 mm/s results in fully amorphized API.

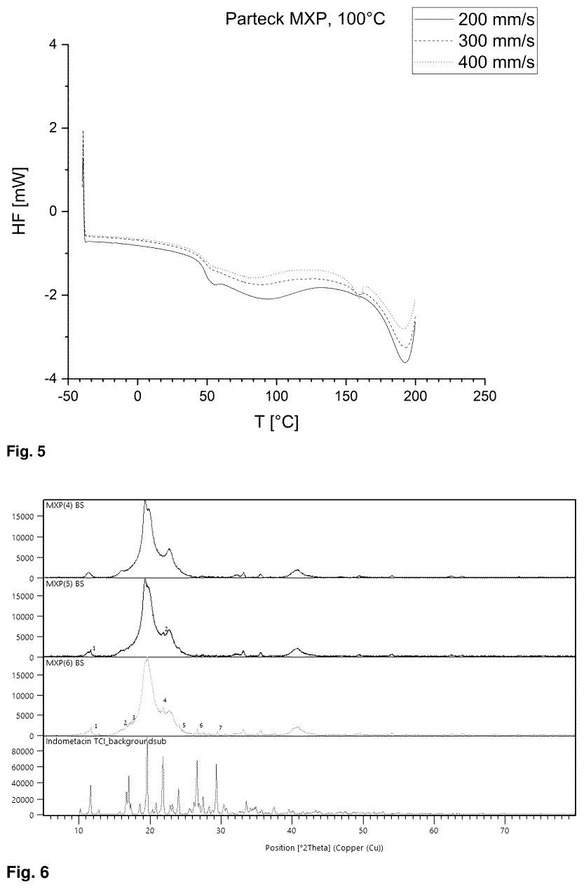

FIG. 5 shows the DSC thermogram for batches printed at 100° C. with PVA 4-88.

FIG. 6 shows diffractograms for batches printed at 100° C. and Indomethacin with PVA 4-88 (the number in parentheses reflects the batch number).

Traces of crystalline API remaining for batch printed at 125° C. and 400 mm/s whereas 200 and 300 mm/s results in fully amorphized API.

FIG. 7 shows the DSC thermogram for batches printed at 125° C. with PVA 4-88.

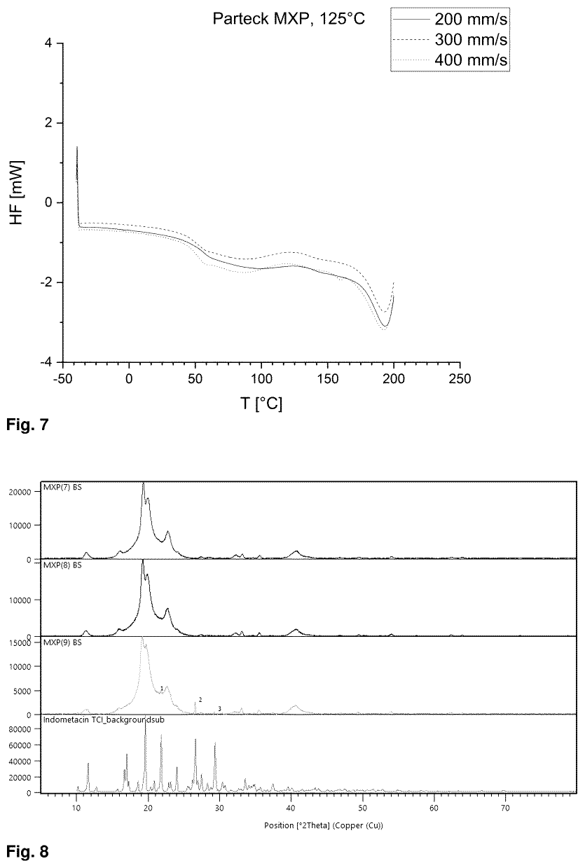

FIG. 8 shows diffractograms for batches printed at 125° C. and Indomethacin with PVA 4-88 (the number in parentheses reflects the batch number).

### Example 2: SLS Printing with PVA 3-82

The preparation of powder formulation was performed according to Example 1. As polyvinyl alcohol, PVA 3-82 was used. The selective laser sintering 3D printing of dosage forms was performed according to Example 1. For Example 2, a layer height of 150 μm was used.

### Characterization of Powder Formulations and 3D Printed Dosage Forms

The methods for characterization of powder formulations and 3D printed dosage forms were performed as described in Example 1.

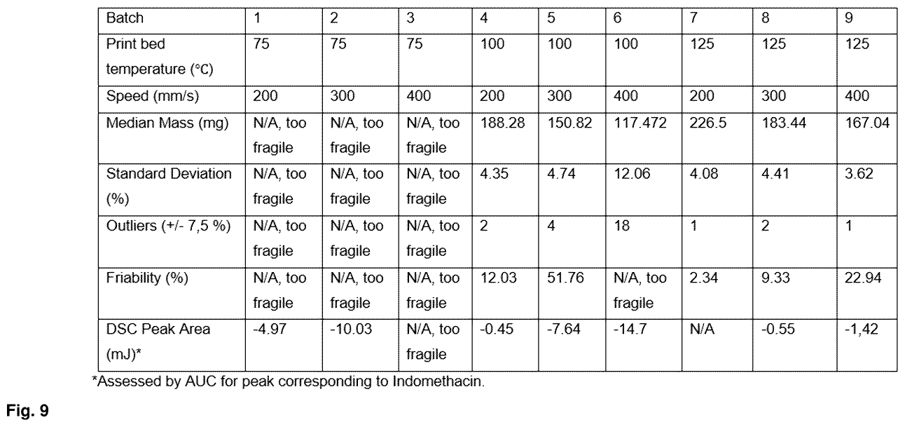

FIG. 9 shows process parameters and characteristics of 3D printed dosage forms.

Traces of crystalline API remaining for batch printed at 75° C. and 300 mm/s whereas 200 mm/s results in fully amorphized API.

In the figures PVA 3-82 is referred to as Polymer 1.

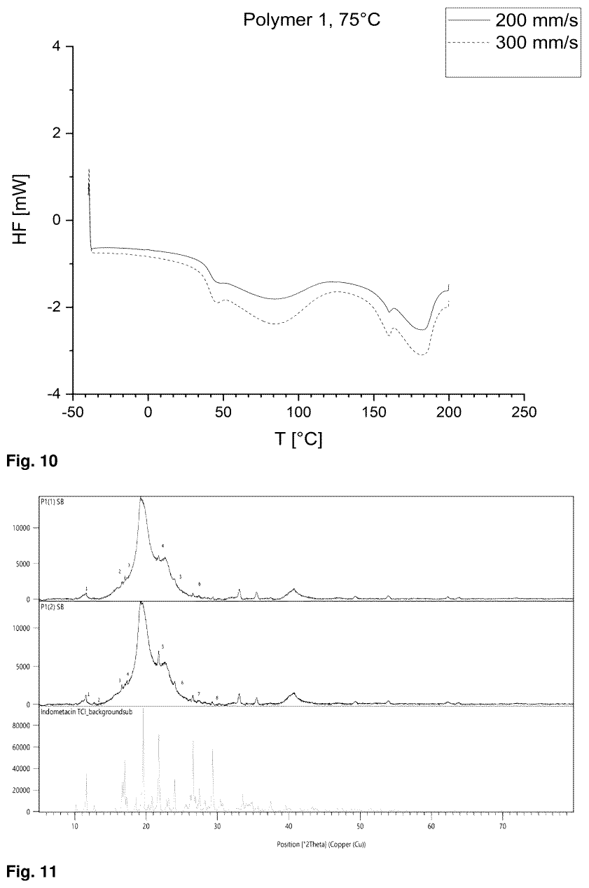

FIG. 10 shows the DSC thermogram for batches printed at 75° C. with PVA 3-82.

FIG. 11 shows diffractograms for batches printed at 75° C. and Indomethacin with PVA 3-82 (the number in parentheses reflects the batch number).

Traces of crystalline API remaining for batch printed at 100° C. and 300 and 400 mm/s whereas 200 mm/s results in fully amorphized API.

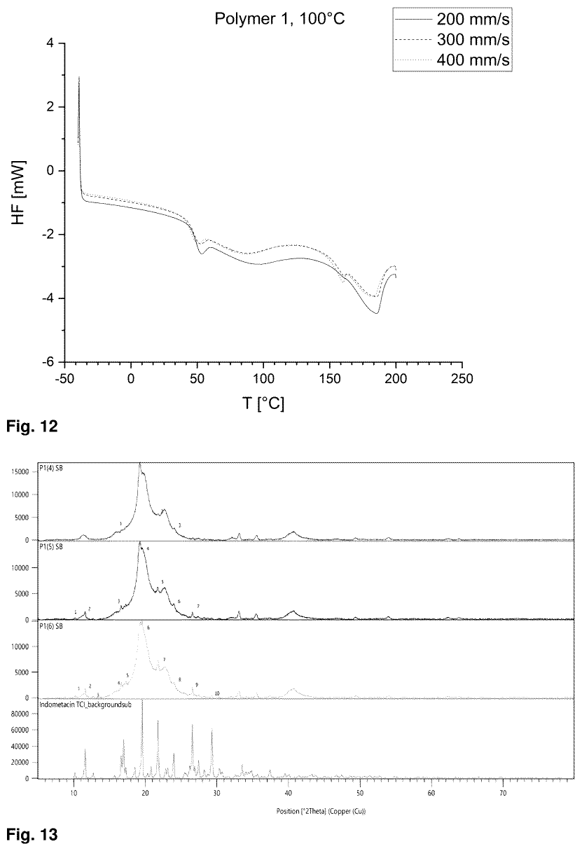

FIG. 12 shows the DSC thermogram for batches printed at 100° C. with PVA 3-82.

FIG. 13 shows diffractograms for batches printed at 100° C. and Indomethacin with PVA 3-82 (the number in parentheses reflects the batch number).

Traces of crystalline API remaining for batch printed at 125° C. and 400 mm/s whereas 200 and 300 mm/s results in fully amorphized API.

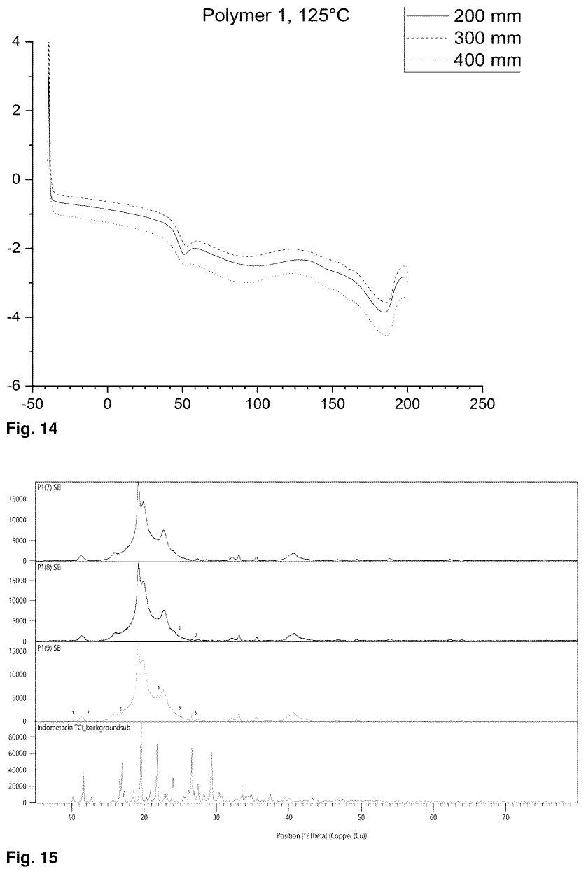

FIG. 14 shows the DSC thermogram for batches printed at 125° C. with PVA 3-82.

FIG. 15 shows diffractograms for batches printed at 125° C. and Indomethacin (the number in parentheses reflects the batch number) with PVA 3-82.

### Example 3: SLS Printing with PVA 5-74

The preparation of powder formulation was performed according to Example 1. As polyvinyl alcohol, PVA 5-74 was used. The selective laser sintering 3D printing of dosage forms was performed according to Example 1. For Example 3, a layer height of 150 μm was used.

### Characterization of Powder Formulations and 3D Printed Dosage Forms

The methods for characterization of powder formulations and 3D printed dosage forms were performed as described in Example 1.

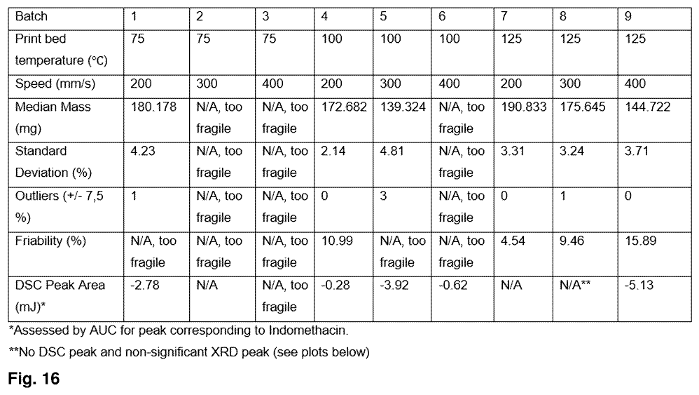

FIG. 16 shows process parameters and characteristics of 3D printed dosage forms.

Traces of crystalline API remaining for batch printed at 75° C. and 200 mm/s. In the figures PVA 5-74 is referred to as Polymer 2.

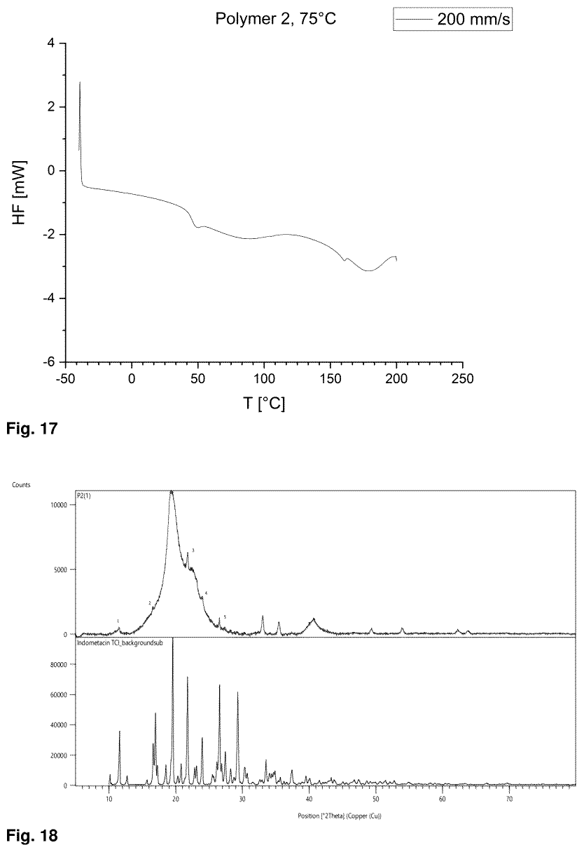

FIG. 17 shows the DSC thermogram for batch printed at 75° C. and 200 mm/s with PVA 5-74.

FIG. 18 shows diffractograms for batch printed at 75° C. (200 mm/s) and Indomethacin with PVA 5-74.

Traces of crystalline API remaining for all batches printed at 100° C. However, the DSC and XRD results are mismatched regarding the batches printed at 300 mm/s and 400 mm/s. The size of the API peak on DSC does not correspond to the number of API characterization peaks in the XRD results.

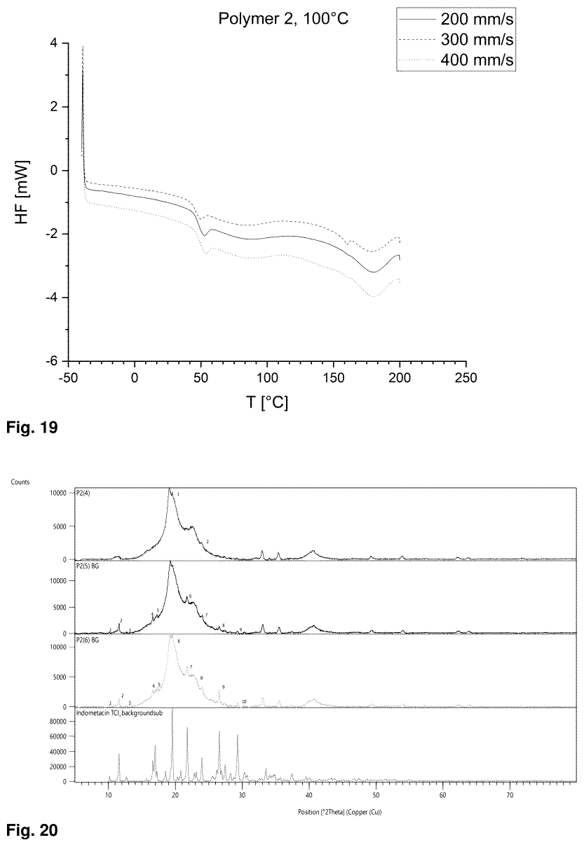

FIG. 19 shows the DSC thermogram for batches printed at 100° C. with PVA 5-74.

FIG. 20 shows diffractograms for batches printed at 100° C. and Indomethacin with PVA 5-74 (the number in parentheses reflects the batch number).

Traces of crystalline API remaining for batch printed at 125° C. and 400 mm/s whereas 200 mm/s results in no trace of crystallinity. Regarding the batch printed at 300 mm/s, according to the DSC, there is no trace of crystallinity of the API. In the XRD results for the batch printed at 300 mm/s, there is one small peak, however, which may be indicative of some crystallinity.

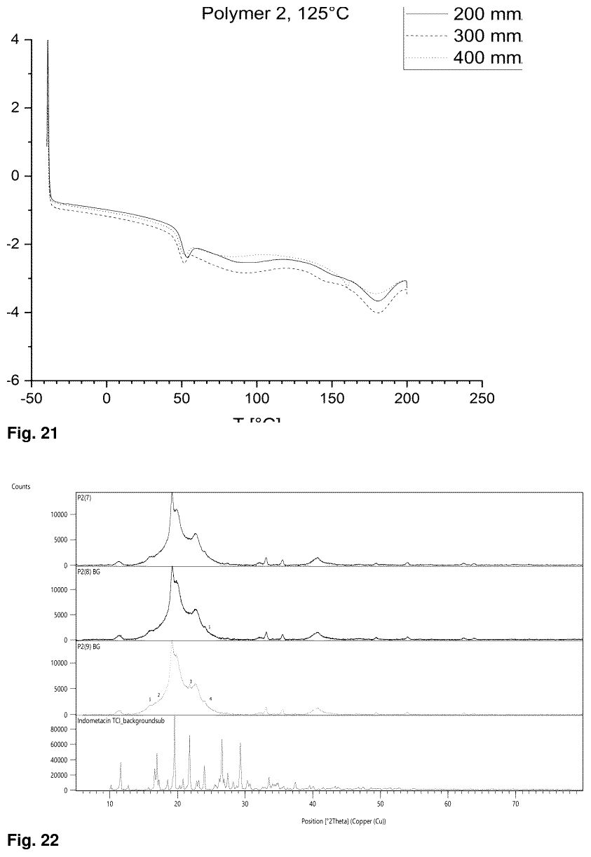

FIG. 21 shows the DSC thermogram for batches printed at 125° C. with PVA 5-74.

FIG. 22 shows diffractograms for batches printed at 125° C. and Indomethacin with PVA 5-74 (the number in parentheses reflects the batch number).

### Example 4: Dissolution Tests

### Preparation of Powder Formulation

PVA 3-82 and PVA 5-74 were prepared alongside PVA 4-88, Kollidon VA64®, and Plasdone™ S-630 in different formulations for selective laser sintering (SLS). Each polymer was prepared in a formulation calculated by weight of 88.5% polymer, 1% pigment, 0.5% silicon dioxide, and 10% indomethacin. The formulations were prepared by weighing, manually mixing, and sieving using a 315 μm stainless-steel test sieve (VWR International AB, Stockholm, Sweden). The sieved formulations were mixed again using a Turbula shaker (Turbula T2F shaker, Glen Mills, Inc., Clifton, NJ, US) for 20 min. Pigment and colloidal silica were added to the formulations to enhance the laser energy absorption of the powders and to improve powder flowability during the layer application process. Then, the mixed powder was heat-treated at 70° C. in a thermostat (Incucell®), BMT Medical Technology s.r.o., Brno, Czech Republic) overnight and mixed again using a Turbula shaker.

### Selective Laser Sintering 3D Printing of Dosage Forms

Prior to printing, the tablets were created in Fusion 360 (Student Edition, Autodesk, USA) and subsequently uploaded as an STL-file to Sintratec Central 1.2.7 (Sintratec AG, Brugg, Switzerland). Batches of 36 tablets were created and arranged in the printing chamber. Then, the following parameters were set for all formulations: 50 μm perimeter offset, 50 μm hatching space, 150 μm hatching offset, and 3 perimeter paths. The sintering was carried out using a 2.3 W diode (λ=455 nm) in accordance with the template models given in the STL-file in a layer-by-layer fashion. The tablets were printed flat to the build platform, using a layer height of 150 μm for Kollidon VA64@ and 125 μm for all other polymers. Specific values for the laser scanning speed were chosen when printing the different batches and each speed was used at three different printing temperatures. The batches created with PVA 3-82, PVA 5-74, and PVA 4-88 were printed at 75, 100, and 125° C., while the batches created with Kollidon VA64@ and Plasdone™ S-630 were printed at 75, 100, and 112.5° C. For the laser scan speed, the three chosen speeds were 200, 300, and 400 mm/s. The finished batches were collected from the build platform at the end of the printing process by sieving. The tablets were additionally de-dusted using pressurized air to remove excess powder and stored in sealed containers for further analysis.

### Dissolution Tests

Dissolution tests were carried out using a Sotax AT7 Smart Dissolution Tester (Aesch, Switzerland). In-vitro drug release profiles for the 3D printed tablets (n=3) were performed in 500 mL of Simulated Gastric Fluid (SGF: 800 ml 1M HCl, 20 g NaCl ad 10 L, pH 1.2.) at 37±0.5° C. and 50 rpm using a sinker to weigh down the tablets. The drug concentration in the dissolution media was determined with high performance liquid chromatography (HPLC) (Agilent 1260 Infinity II, Agilent Technologies, Inc., Santa Clara, USA) on 10 μL of filtered sample (0.45 μm PTFE filters, VWR International GmbH). The HPLC assays were performed using a mobile phase composition of acetonitrile and phosphate buffer (0.01M NaH₂PO*H₂O 1.38 g/L+0.01M Na₂HPO₄ 1.41 g/L) in 1 to 1 ratio. Samples were injected into a Supelcosil LC-18 column (30×4 mm, 5 μm) at a flow-rate of 1 mL min⁻¹ and at 40° C. and the eluent analyzed at 254 nm. These dissolution tests were performed on the batch for each polymer with the best characteristics in terms of weight distribution, PXRD, DSC, and friability. For PVA 3-82, PVA 5-74, and PVA 4-88, these were the batches printed at 125° C. and 200 mm/s. For Kollidon VA64® and Plasdone™ S-630, these were the batches printed at 112.5° C. and 200 mm/s.

FIG. 23 shows the dissolution results for the best batches from each formulation drug loading test.

1. A process for selective laser sintering of sinter powder to form a pharmaceutical dosage form, comprising achieving said process by a selective laser sintering apparatus, wherein the sinter powder comprises at least one active pharmaceutical ingredient and polyvinyl alcohol having a hydrolysis degree of 70% to 90%, and a viscosity of a 4% solution at 20° C. of 3 mPas to 8 mPas.

2. A method according to claim 1, wherein the polyvinyl alcohol has a hydrolysis degree of 80% to 90%, and a viscosity of a 4% solution at 20° C. of 3 mPas to 4 mPas.

3. A method according to claim 1, wherein the polyvinyl alcohol is PVA 3-80, PVA 3-82, PVA 4-88 or PVA 5-74.

4. A method according to claim 1, wherein the sinter powder further comprises at least one light-absorbing material.

5. Process for producing a pharmaceutical dosage form by selective laser sintering of sinter powder, comprising the steps of
(a) providing a sinter powder comprising at least one active pharmaceutical ingredient and polyvinyl alcohol having a hydrolysis degree of 70% to 90%, and a viscosity of a 4% solution at 20° C. of 3 mPas to 8 mPas, and
(b) operating a selective laser sintering apparatus that selectively fuses layers of the sinter powder to produce the pharmaceutical dosage form.

6. Process according to claim 5, wherein the polyvinyl alcohol has a hydrolysis degree of 80% to 90%, and a viscosity of a 4% solution at 20° C. of 3 mPas to 4 mPas.

7. Process according to claim 5, wherein the sinter powder further comprises at least one light-absorbing material.

8. Process according to claim 5, wherein the particle size of the sinter powder has a D50 of 200 μm or lower.

9. A sinter powder for selective laser sintering, comprising at least one active pharmaceutical ingredient and polyvinyl alcohol having a hydrolysis degree of 70% to 90%, and a viscosity of a 4% solution at 20° C. of 3 mPas to 8 mPas.

10. A sinter powder according to claim 9, wherein the polyvinyl alcohol has a hydrolysis degree of 80% to 90%, and a viscosity of a 4% solution at 20° C. of 3 mPas to 4 mPas.

11. A sinter powder according to claim 9, further comprising at least one light-absorbing material.

12. A pharmaceutical dosage form obtainable by a process according to claim 5.

13. A pharmaceutical dosage form produced by selective laser sintering of the sinter powder according to claim 9, wherein the sinter powder comprises at least one active pharmaceutical ingredient and polyvinyl alcohol having a hydrolysis degree of 70% to 90%, and a viscosity of a 4% solution at 20° C. of 3 mPas to 8 mPas.

14. A pharmaceutical dosage form according to claim 13, wherein the polyvinyl alcohol has a hydrolysis degree of 80% to 90%, and a viscosity of a 4% solution at 20° C. of 3 mPas to 4 mPas.

15. A pharmaceutical dosage form according to claim 13, wherein the sinter powder further comprises at least one light-absorbing material.
